(19) United States
(12) Patent Application Publication
(10) Pub. No.: US 2025/0125048 A1
(43) Pub. Date: Apr. 17, 2025

(54) Method for Training Model, Method for Generating Treatment Plan, and Medium

(71) Applicants: OUR UNITED CORPORATION, Shaanxi (CN); SHENZHEN OUR NEW MEDICAL TECHNOLOGIES DEVELOPMENT CO., LTD., Shenzhen (CN); OUR SMD Medical (Wuhan) Co., Ltd., Wuhan (CN)

(72) Inventors: Peng Guo, Shaanxi (CN); Jinsheng Li, Shaanxi (CN)

(21) Appl. No.: 18/385,811

(22) Filed: Oct. 31, 2023

(30) Foreign Application Priority Data
Oct. 16, 2023 (CN) 202311337930.1

(51) Int. Cl. G16H 50/20; G16H 30/40

(52) U.S. Cl. CPC G16H 50/20; G16H 30/40

(57) ABSTRACT

A method for training a deep reinforcement learning model includes performing a training process. The training process including the following operations acquiring initial dose distribution state data of an objective target volume; determining, based on the initial dose distribution state data of the objective target volume and current policy data of the actor network and the critic network, output data of a plurality of sub-threads in parallel by using the plurality of sub-threads; and updating the current policy data of the actor network and the critic network based on the output data of each sub-thread of the plurality sub-threads in sequence, so as to complete a current training for the deep reinforcement learning model; and iterating the training process until a count of training the deep reinforcement learning model reaches a preset count, so as to obtain the deep reinforcement learning model that has been trained.

### CROSS-REFERENCE TO RELATED APPLICATION

This application claims priority to Chinese Patent Application No. 202311337930.1, filed Oct. 16, 2023, the disclosure of which is hereby incorporated by reference in its entirety.

### BACKGROUND OF THE INVENTION

### Field of the Invention

The present disclosure relates to the field of medical technology, and in particular, to the field of radiotherapy, for example, to a method for training a model, a method for generating a treatment plan, and a medium.

### Description of Related Art

In the field of medical technology, the radiotherapy is one of the important means to treat tumors. Before using the radiotherapy for treating a to-be-treated subject, it is usually necessary to design a treatment plan in advance.

At present, the design for a more reasonable treatment plan usually relies on a physician to repeatedly adjust the treatment plan (manually) based on their own experience and professional skills. Therefore, the existing methods for generating a treatment plan require a high level of clinical experience from the physicians, and the treatment plan requires continuous trial and error, which is time-consuming, laborious and inefficient.

### SUMMARY OF THE INVENTION

The present disclosure provides a method for training a model, a method for generating a treatment plan, and a medium.

One aspect of the present disclosure provides a method for training a deep reinforcement learning model for generating a treatment plan. The deep reinforcement learning model is configured to include an actor network and a critic network. The method includes: performing a training process, wherein the training process includes the following operations: acquiring initial dose distribution state data of an objective target volume; determining, based on the initial dose distribution state data of the objective target volume and current policy data of the actor network and the critic network, output data of each sub-thread of a plurality of sub-threads in parallel by using the plurality of sub-threads; and updating the current policy data of the actor network and the critic network based on the output data of each sub-thread of the plurality sub-threads in sequence, so as to complete a current training for the deep reinforcement learning model; and iterating the training process until a count of training the deep reinforcement learning model reaches a preset count, so as to obtain the deep reinforcement learning model that has been trained; where the output data of the sub-thread includes: a final dose distribution of the objective target volume, multiple dose distribution state data of the objective target volume, a plurality of target parameters corresponding to the multiple dose distribution state data respectively, a plurality of predicted values corresponding to the plurality of target parameters respectively, a plurality of actual rewards corresponding to the plurality of target parameters respectively, a predicted value of the objective target volume, and an actual reward of the objective target volume.

Another aspect of the present disclosure provides a method for generating a treatment plan. The method includes: acquiring image data of a to-be-treated target volume and contour data of the to-be-treated target volume; determining, based on the image data and the contour data, dose distribution state data of the to-be-treated target volume; inputting the dose distribution state data of the to-be-treated target volume into a deep reinforcement learning model, so as to obtain a target parameter of the to-be-treated target volume; the deep reinforcement learning model is trained by the method for training the deep reinforcement learning model according to the above aspect.

Yet another aspect of the present disclosure provides an electronic device. The electronic device includes at least one processor, and a storage connected with the at least one processor. The storage stores an instruction that can be executed by the at least one processor, when the instruction is executed by the at least one processor, causing the at least one processor to implement the method for training the deep reinforcement learning model according to the above aspect.

Yet another aspect of the present disclosure provides a non-transitory computer readable storage medium having stored a computer program thereon, where the program, upon being executed by a processor, implements the method for training the deep reinforcement learning model according to the above aspect.

In the technical solutions provided in the present disclosure, a deep reinforcement learning model for generating a treatment plan may be trained by a plurality of sub-threads in parallel. Due to the parallel trial and error capability of the plurality of sub-threads, the speed of training the model is improved. Due to the fact that the deep reinforcement learning model conforms to the characteristics of the design for the treatment plan with Gamma knife, and a processor in a computer system is able to repeat the trial and error process, target parameters with better performance may be automatically generated through the deep reinforcement learning model, resulting in generating a better treatment plan based on the target parameters with better performance. Thus, the dependence on clinical experience may be reduced, the effect of treatment plan may be improved without the need for manual setting of target parameters, and further, the efficiency for generating a treatment plan by a physician is improved.

It should be understood that the contents described in the section is not intended to identify keys or important features of the embodiments of the present disclosure, nor is it used to limit the scope of the present disclosure. The other features of the present disclosure will be easily understood through the following description.

### BRIEF DESCRIPTION OF THE DRAWINGS

The drawings are intended for a better understanding of the embodiments in the present disclosure, and do not constitute the limitation of the present disclosure.

FIG. 1 is a schematic diagram of an exemplary application scenario of a method for training a deep reinforcement learning model for generating a treatment plan, and a method for generating a treatment plan according to some embodiments.

FIG. 2 is a flow chart of an exemplary method for training a deep reinforcement learning model for generating a treatment plan according to some embodiments.

FIG. 3A is another flow chart of an exemplary method for training a deep reinforcement learning model for generating a treatment plan according to some embodiments.

FIG. 3B is another flow chart of sub-thread 1 or 2 in FIG. 3A according to some embodiments.

FIG. 4A is yet another flow chart of an exemplary method for training a deep reinforcement learning model for generating a treatment plan according to some embodiments.

FIG. 4B is another flow chart of sub-thread 1 or 2 in FIG. 4A according to some embodiments.

FIG. 5 is a flow chart of an exemplary method for generating a treatment plan according to some embodiments.

FIG. 6 is a block diagram of an exemplary electronic device implementing some embodiments.

### DESCRIPTION OF THE INVENTION

Exemplary embodiments of the present disclosure are described below with reference to the drawings, in which various details of the embodiments of the present disclosure are included to facilitate understanding, and these embodiments should be considered as exemplary only. Therefore, those skilled in the art should realize that various changes and modifications can be made to the embodiments described herein without departing from the scope of the present disclosure. Similarly, for clarity and conciseness, description of well-known functions and structures have been omitted from the following description.

In the technical solution in the present disclosure, the collection, storage, usage, processing, transmission, provision, and disclosure of user personal information comply with relevant laws and regulations, and do not violate public order and good customs.

Before a detailed description on the present disclosure is provided, the application scenarios involved in the embodiments of the present disclosure are described first.

A method for training a deep reinforcement learning model for generating a treatment plan, a method for generating a treatment plan, a device, and a medium provided in the embodiments of the present disclosure may be applied in the field of medical technology, and in particular, in the scenarios of the clinical radiotherapy, such as a scenario for designing a treatment plan with Gamma knife.

Before radiotherapy for treating a to-be-treated subject is adopted, it is usually necessary to design a treatment plan in advance.

At present, the design for a more reasonable treatment plan usually relies on a physician to repeatedly adjust the treatment plan manually based on their own experience and professional skills. Therefore, the existing methods for generating a treatment plan require a high level of clinical experience from the physician, and the treatment plan requires continuous trial and error, which is time-consuming, laborious, and inefficient.

Based on this, the embodiments of the present disclosure provide a method for training a deep reinforcement learning model for generating a treatment plan, a method for generating a treatment plan, a device, and a medium, where the deep reinforcement learning model includes an actor network and a critic network.

The method for training a deep reinforcement learning model includes: performing a training process, the training process including the following operations: acquiring initial dose distribution state data of an objective target volume; determining, based on the initial dose distribution state data of the objective target volume and current policy data of the actor network and the critic network, output data of each sub-thread of a plurality of sub-threads in parallel by using the plurality of sub-threads; and updating the current policy data of the actor network and the critic network based on the output data of each sub-thread of the plurality sub-threads in sequence, so as to complete a current training for the deep reinforcement learning model; and iterating the training process until a count of training the deep reinforcement learning model reaches a preset count, so as to obtain the deep reinforcement learning model that has been trained;
where the output data of the sub-thread includes: a final dose distribution of the objective target volume, multiple dose distribution state data of the objective target volume, a plurality of target parameters corresponding to the multiple dose distribution state data respectively, a plurality of predicted values corresponding to the plurality of target parameters respectively, a plurality of actual rewards corresponding to the plurality of target parameters respectively, a predicted value of the objective target volume, and an actual reward of the objective target volume.

As set forth above, the deep reinforcement learning model for generating a treatment plan is trained by a plurality of sub-threads in parallel in the present disclosure. Due to the parallel trial and error capability of the plurality of sub-threads, the speed of training the model is improved. Due to the fact that the deep reinforcement learning model conforms to the characteristics of the design for the treatment plan with Gamma knife, and a processor in a computer system is able to repeat the trial and error process, target parameters with better performance may be automatically generated through the deep reinforcement learning model, resulting in generating a better treatment plan based on the target parameters with better performance. Thus, the dependence on clinical experience may be reduced, the effect of the treatment plan may be improved without the need for manual setting of target parameters, and further, the efficiency for generating a treatment plan is improved.

The methods provided by the present disclosure mainly relate to a method for training a deep reinforcement learning model for generating a treatment plan, and a method for generating a treatment plan. The application scenarios of the above two methods are introduced below.

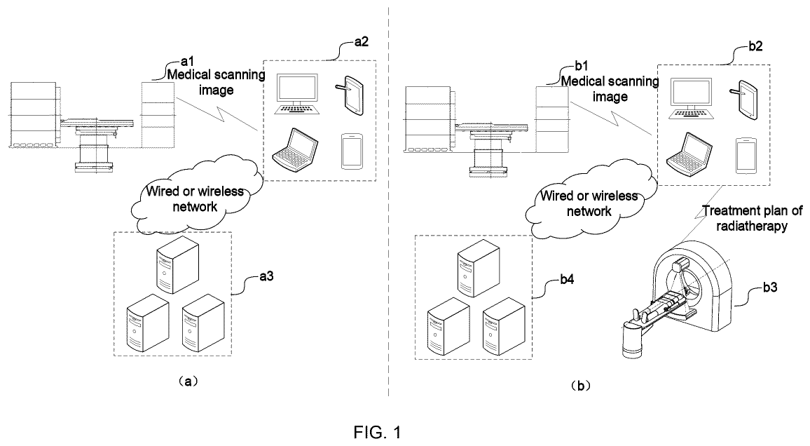

(a) in FIG. 1 is a schematic diagram of an exemplary application scenario of a method for training a model according to some embodiments. As shown in (a) in FIG. 1, an image scanning device a1 and an electronic device a2 are included in the application scenario.

The image scanning device a1 is a device configured to scan and display a tumor and surrounding normal tissues of a to-be-treated subject. In some embodiments, the imaging scanning device a1 may be at least one of the following devices: a computed tomography (CT) device, an emission computed tomography (ECT) device, a magnetic resonance imaging (MRI) device, a positron emission tomography (PET) device, or an ultrasound inspection device.

In some embodiments, the image scanning device a1 may be configured to acquire a medical scanning image (e.g., image data of a target volume) of the to-be-treated subject and upload the medical scanning image of the to-be-treated subject to the electronic device a2. Thus, the electronic device a2 performs a method for training a model based on the medical scanning image.

The electronic device a2 is a device configured to train a deep reinforcement learning model. In some embodiments, the electronic device a2 may be at least one of the following devices: a smartphone, a smart watch, a desktop computer, a portable computer, a virtual reality terminal, an augmented reality terminal, a wireless terminal, or a laptop portable computer.

In some embodiments, the electronic device a2 may run a computer system that includes a processor for implementing the method for training the deep reinforcement learning model.

In some embodiments, a server a3 is also included in the application scenario. In some embodiments, the server a3 is configured to provide a background communication service for the image scanning device a1 and the electronic device a2.

The server a3 may be an independent physical server, a distributed file system or a server cluster composed of multiple physical servers, or at least one of cloud servers that can provide the following basic cloud computing services: providing cloud services, cloud databases, cloud computing, cloud functions, cloud storage, network services, cloud communication, middleware services, domain name services, security services, content distribution networks, mega data or an artificial intelligence platform, which may not be limited in the embodiments of the present disclosure. In some embodiments, a number of the above server a3 is one or more, which is not limited in the embodiments of the present disclosure. Of course, the server a3 can also include other functions to provide more comprehensive and diversified services.

(b) in FIG. 1 is a schematic diagram of an exemplary application scenario of a method for generating a treatment plan according to some embodiments. As shown in (b) in FIG. 1, an image scanning device b1, an electronic device b2, and a radiotherapy device b3 are included in the application scenario.

A form of the image scanning device b1 is similar to that of the image scanning device a1 in (a) in FIG. 1, and may refer to the description on the image scanning device a1, which will not be repeated herein.

In some embodiments, the image scanning device b1 is configured to acquire a medical scanning image (e.g., image data of a to-be-treated target volume) of the to-be-treated subject and upload the medical scanning image of the to-be-treated subject to the electronic device b2. Thus, the electronic device b2 performs a method for generating a treatment plan based on the medical scanning image.

The electronic device b2 is a device configured to generate a treatment plan by using the deep reinforcement learning model.

A form of the electronic device b2 is similar to that of the electronic device a2 in (a) in FIG. 1, and may refer to the description on the description on the electronic device a2, which will not be repeated herein.

In some embodiments, the electronic device b2 may run a computer system that includes a processor for implementing a method for generating a treatment plan using the deep reinforcement learning model.

The radiotherapy device b3 is a device used for radiotherapy. In some embodiments, the radiotherapy device b3 may be at least one of the following devices: a Gamma knife, a linear accelerator, a neutron knife, or an X-ray therapy machine.

In some embodiments, the radiotherapy device b3 is configured to receive a treatment plan from the electronic device b2, and perform radiotherapy on the to-be-treated subject based on the treatment plan.

In some embodiments, a server b4 is also included in the application scenario. In some embodiments, the server b4 is configured to provide a background communication service for the image scanning device b1, the electronic device b2, and the radiotherapy device b3 mentioned above.

A form of the server b4 is similar to that of the server a3 in (a) in FIG. 1, and may refer to the description on the description on the server a3, which will not be repeated herein.

Based on the application scenario shown in (a) in FIG. 1, the method for training the model provided in the present disclosure would be introduced first.

The method for training the deep reinforcement learning model for generating a treatment plan provided in the embodiments of the present disclosure may be applied to the electronic device a2 in (a) in FIG. 1, which may run a computer system including a processor for implementing the method for training the deep reinforcement learning model for generating a treatment plan.

The deep reinforcement learning model includes an actor network and a critic network.

The actor network includes an action decision parameter. The action decision parameter is used to characterize a probability of a target parameter that is available for being selected under a certain dose distribution state. If the probability of the target parameter being selected is higher, it means that it is more suitable for selecting the corresponding target parameter under a certain dose distribution state.

For example, when the target parameter is a size of a target, under a certain dose distribution state, sizes of target that are available for being selected are Ø4, Ø8, and Ø10, respectively, and thus, corresponding probabilities of sizes of target that are available for being selected are 10%, 20%, and 70%. Thus, in this certain dose distribution state, the optimal size of a target is selected as Ø10.

The critic network may include a predicted decision parameter. The predicted decision parameter is used to determine a predicted value corresponding to each target parameter. The predicted value is used to characterize a quality of a selection of a target parameter under a certain dose distribution state.

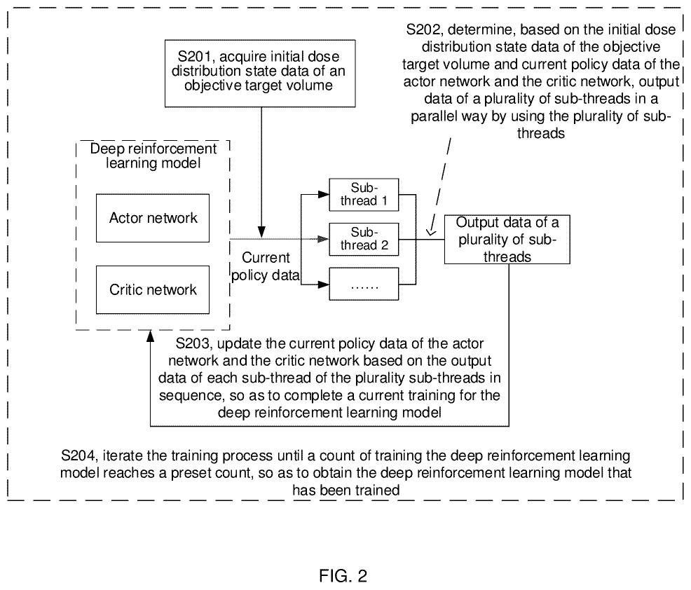

FIG. 2 is a flow chart of an exemplary method for training a deep reinforcement learning model for generating a treatment plan according to some embodiments of the present disclosure. As shown in FIG. 2, the method includes steps S201, S202, S203, and S204.

In S201, initial dose distribution state data of an objective target volume is acquired.

The objective target volume includes a to-be-treated area of a to-be-treated subject. A number of the objective target volume may be one or multiple. When the number of the objective target volume is multiple, the multiple target volumes may be in different shapes.

In some embodiments, the to-be-treated subject may be a phantom, a human body, or an animal. The objective target volume may be, for example, a tumor of the to-be-treated subject. The objective target volume may also be referred to as a planning target volume (PTV).

For example, as shown in FIG. 2, image data and contour data of different target volumes may include: image data and contour data of a target volume 1, image data and contour data of a target volume 2, etc.

For example, in combination with (a) in FIG. 1, the image scanning device a1 may acquire image data of the to-be-treated subject and transmit the image data to the electronic device a2.

In some embodiments, the electronic device a2, after receiving the image data, may outline the image data to obtain contours (i.e., contour data) of different target volumes of the to-be-treated subject in the image data.

For example, the contour data may be a contour of a tumor.

In some embodiments, the electronic device a2 may also be connected to a third-party software program. This third-party software program may be configured to outline the image data to obtain the contour data.

In some embodiments, when the contour of the image data is outlined, the outlining operation may be performed by a physician on the electronic device a2. The electronic device a2 may respond to the outlining operation performed by the physician to obtain the contour data.

In some embodiments, the electronic device a2 may also automatically outline the contours of different target volumes of the to-be-treated subject in the image data through an outlining software.

After the image data and the contour data of the objective target volume is determined, the processor in the electronic device a2 may determine the initial dose distribution state data of the objective target volume based on the image data and the contour data of the objective target volume.

The initial dose distribution state data refers to dose distribution state data of the objective target volume when no target is placed.

The dose distribution state data includes: mask data of the objective target volume, a dose distribution of the objective target volume, a volume of an area in the objective target volume with an insufficient dose distribution, and a volume of an area in the objective target volume with an overflow dose.

Due to the fact that the initial dose distribution state data refer to the dose distribution state data of the objective target volume when no target is placed, dose distribution, a volume of an area in the objective target volume with an insufficient dose distribution, and a volume of an area in the objective target volume with an overflow dose in the initial dose distribution state data are all 0. In this case, the processor in the electronic device a2 can determine the initial dose distribution state data of the objective target volume only by determining the mask data of the objective target volume according to the image data and the contour data of the objective target volume.

Specifically, the image data of the objective target volume may include image data of the objective target volume and image data of an organ at risk (OAR).

The OAR refers to a normal organ around the objective target volume, i.e., an organ that has not undergone pathological changes.

The processor in electronic device a2 may process the image data and the contour data of the objective target volume to produce the mask data of the objective target volume. In a process of producing the mask data, the processor in the electronic device a2 may perform different operations on areas corresponding to the objective target volume and OARs based on the contour data, respectively, thereby distinguishing the areas corresponding to the objective target volume and the OARs.

At the same time, a searching space for the positions of targets (i.e., positions for placing the targets) may be limited within the area corresponding to the objective target volume, thus avoiding the positions of target falling into the areas corresponding to OARs or areas corresponding to other tissues, thereby avoiding treatment damage to the areas corresponding to OARs or the areas corresponding to other tissues. Moreover, limiting the searching space for the positions of the targets within the areas corresponding to the objective target volume may reduce searching space for a specific shape in the objective target volume, thus improving the speed of training the model and generating the treatment plan.

For example, the processor in the electronic device a2 may construct a three-dimensional matrix with a uniform size. Image parameters of the areas corresponding to the objective target volume may be set as 1, image parameters of the areas corresponding to the OARs may be set as (−1), and image parameters of the areas corresponding to other tissues may be set as 0. Thus, mask data where the areas corresponding to the objective target volume and the areas corresponding to OARs are distinguished may be obtained.

In S202, output data of each sub-thread of a plurality of sub-threads is determined in parallel by using the plurality of sub-threads based on the initial dose distribution state data of the objective target volume and current policy data of the actor network and the critic network.

For example, as shown in FIG. 2, the plurality of sub-threads may include: sub-thread 1, sub-thread 2, etc.

The output data of the sub-thread includes: a final dose distribution of the objective target volume, multiple dose distribution state data of the objective target volume, a plurality of target parameters corresponding to the multiple dose distribution state data respectively, a plurality of predicted values corresponding to the plurality of target parameters respectively, a plurality of actual rewards corresponding to the plurality of target parameters respectively, a predicted value of the objective target volume, and an actual reward of the objective target volume.

For example, the processor of the electronic device a2, after determining the initial dose distribution state data of the objective target volume, may input the initial dose distribution state data into each sub-thread, for each sub-thread to develop a complete treatment plan for the objective target volume based on the initial dose distribution state data and current policy data of the actor network and critic network, and further, the output data of each sub-thread is determined.

When a first training on the deep reinforcement learning model is performed, the current policy data of the actor network and the critic network is initial policy data of the actor network and the critic network. Each sub-thread may develop the complete treatment plan for the objective target volume based on the initial policy data of the actor network and the critic network and the initial dose distribution state data of the objective target volume, and further, first output data of each sub-thread is determined.

When a current training is not the first training performed on the deep reinforcement learning model, each sub-thread develops a complete treatment plan for the objective target volume based on the current policy data of the actor network and the critic network, and the initial dose distribution state data, and further, current output data of each sub-thread is determined.

Due to the fact that a generation process of a treatment plan with Gamma knife is a process for placing targets in sequence, a dose distribution state of the objective target volume would change after the placement of a target is completed. Therefore, during the generation process of a complete treatment plan for the objective target volume, the dose distribution state of the objective target volume would change with the placement of one target after another.

Therefore, in the embodiments of the present disclosure, for each sub-thread, the output data of the sub-thread can only be acquired when a complete treatment plan for the objective target volume is generated.

Specifically, the processor in the electronic device a2, after obtaining the initial dose distribution state data, may input the initial dose distribution state data into the plurality of sub-threads respectively. Due to the fact that the plurality of sub-threads may share the actor network, each sub-thread may output a first type of target parameter selected for the first target based on the initial policy data of the actor network and the initial dose distribution state data of the objective target volume. Then, each sub-thread may call different algorithms (e.g., a target parameter selection algorithm, a target position determination algorithm and a target weight determination algorithm) to determine other types of target parameters, thereby obtaining a target parameter combination for placing the first target. The target parameter combination may include the first type of target parameter selected for the first target output by the actor network, and other types of target parameters selected for the first target determined by calling different algorithms.

Due to the fact that the deep reinforcement learning model may also include the critic network, and each sub-thread may also share the critic network, while determining the first type of target parameter for the first target, the critic network may also determine a predicted value corresponding to the first type of target parameter of the first target based on the initial policy data of the critic network and the initial dose distribution state data of the objective target volume.

After the first target is placed, the dose distribution state of the objective target volume changes. At this time, each sub-thread may also determine the dose distribution of the objective target volume based on the target parameter combination of the first target, and then determine an actual reward corresponding the first type of target parameter of the first target based on the dose distribution of the objective target volume.

Then, each sub-thread may update the dose distribution state data of the objective target volume based on the dose distribution of the objective target volume, and repeat the above processes for placing a target based on the updated dose distribution state data of the objective target volume until a complete treatment plan of the objective target volume is generated. Then, the final dose distribution of the objective target volume, multiple dose distribution state data of the objective target volume, the plurality of target parameters corresponding to the multiple dose distribution state data respectively, the plurality of predicted values corresponding to the plurality of target parameters respectively, the plurality of actual rewards corresponding to the plurality of target parameters respectively, the predicted value of the objective target volume, and the actual reward of the objective target volume are obtained. Thus, the output data of each sub-thread may be obtained.

In S203, the current policy data of the actor network and the critic network is updated based on the output data of each sub-thread of the plurality sub-threads in sequence, so as to complete a current training for the deep reinforcement learning model.

Specifically, obtaining the output data of each sub-thread indicates that a treatment plan by each sub-thread for the objective target volume has been completed. In this case, the current policy data of the actor network and the critic network may be updated based on the output data of each sub-thread of the plurality sub-threads in sequence, so as to complete a current training for the deep reinforcement learning model.

It should be noted that the training process of the deep reinforcement learning model is equivalent to a process of trial and error for generating a treatment plan, that is, a treatment plan generated by each sub-thread may not necessarily be a good plan. Therefore, there are situations where the output data obtained by one or more sub-threads is discarded for the reason that an update condition is not met.

Therefore, in the embodiments of the present disclosure, in the situation where the output data of the plurality of sub-threads does not meet the update condition, there is no need to update the current policy data of the actor network and the critic network. That is, the completion of a training of the deep reinforcement learning model does not indicate that the current policy data of the actor network and the critic network would be updated.

Of course, when the output data of the plurality of sub-threads meets the update condition, the completion of a training of the deep reinforcement learning model indicates that the current policy data of the actor network and the critic network is updated for a plurality of times (i.e., a count for updating the current policy data of the actor network and the critic network is equal to a number of the plurality of sub-threads).

Correspondingly, when the output data of at least one sub-thread (i.e., a portion of the plurality of sub-threads) meets the update condition, the completion of a training of the deep reinforcement learning model indicates that the current policy data of the actor network and the critic network is updated at least once (i.e., a count for updating the current policy data of the actor network and the critic network is equal to a number of the portion of the plurality of sub-threads).

When the current policy data of the actor network and the critic network is updated, the plurality of sub-threads may package the output data of the plurality of sub-threads into a buffer, and transmit the buffer back to the deep reinforcement learning model through a communication pipe, for the deep reinforcement learning model to update the current policy data of the actor network and the critic network, thus completing the current training of the deep reinforcement learning model.

In S204, a trained deep reinforcement learning model is obtained by iterating the training processes S201-S203 until a count of iterating the training process reaches a preset count.

The training processes S201-S203 of the deep reinforcement learning model mentioned above are iterated until the count of iterating the training processes of the deep reinforcement learning model reaches the preset count, and thus, the trained deep reinforcement learning model is obtained.

In some embodiments, a buffer memory may be included in an electronic device. The buffer memory is configured to store the final dose distribution of the objective target volume, the multiple dose distribution state data of the objective target volume, the plurality of target parameters corresponding to the multiple dose distribution state data respectively, the plurality of predicted values corresponding to the plurality of target parameters respectively, the plurality of actual rewards corresponding to the plurality of target parameters respectively, the predicted value of the objective target volume, and the actual reward of the objective target volume (i.e., the output data of each sub-thread) in each stage during the process of updating the current policy data. Subsequently, through a correspondence stored in the buffer memory, various required data determined in each training may be found, and further, a training is performed.

In some embodiments, a number of the objective target volume may one (also referred to as a single objective target volume) or multiple (also referred to as multiple objective target volumes). In this case, the embodiments of the present disclosure provide embodiments of two methods for training a model: a method for training a model implemented in a scenario with a single target volume and a method for training a model implemented in a scenario with multiple target volumes.

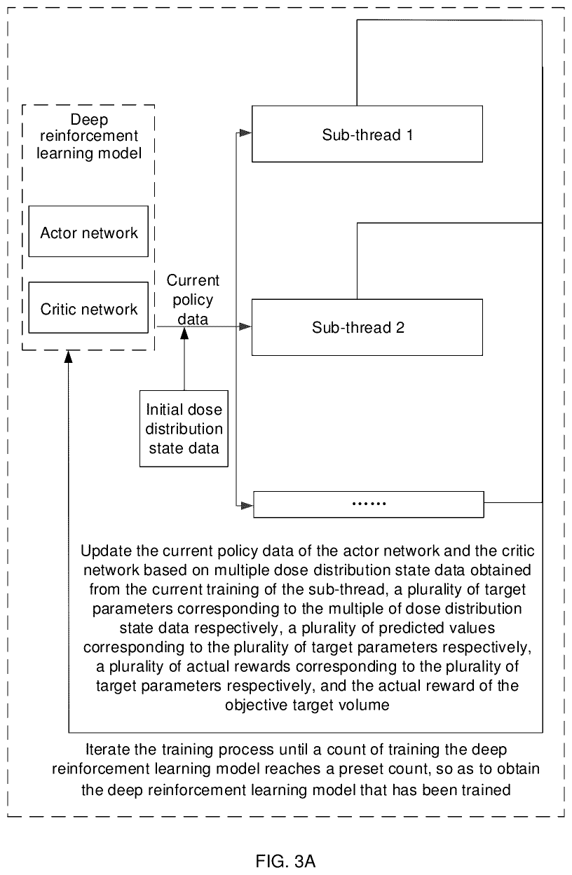

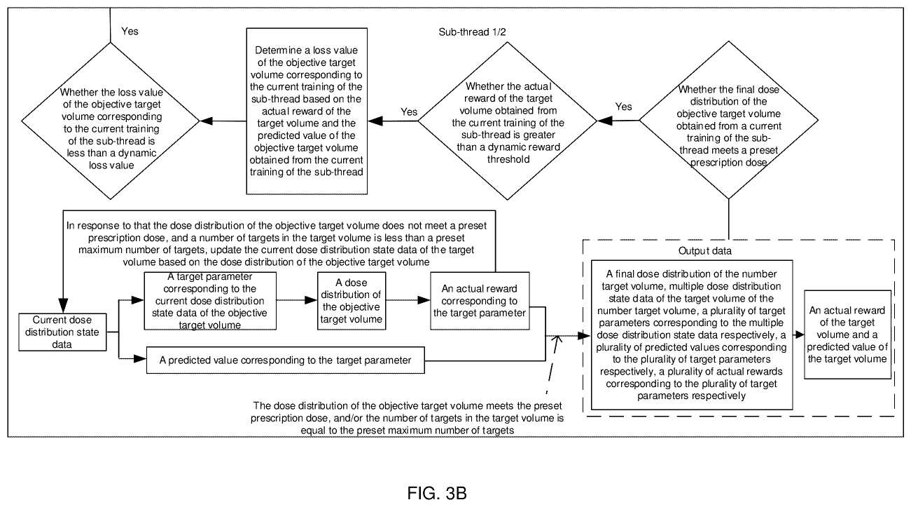

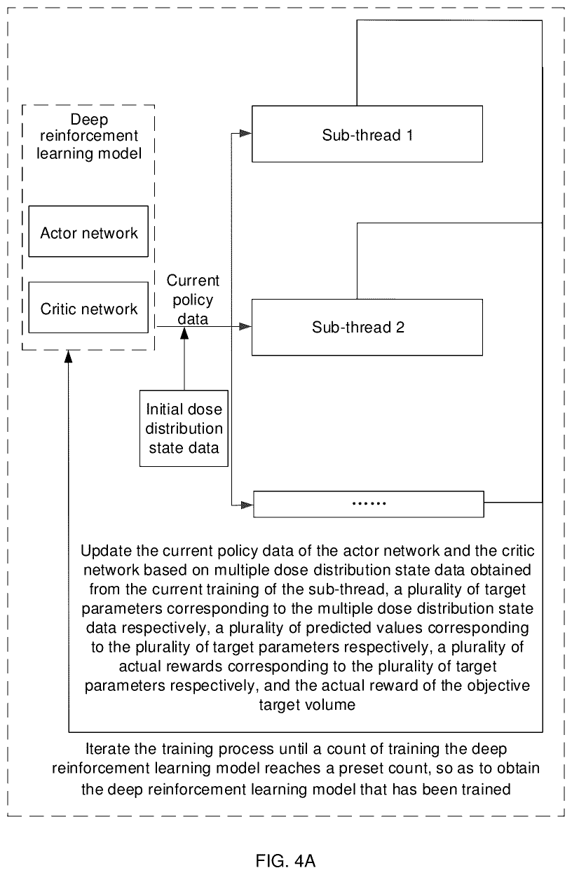

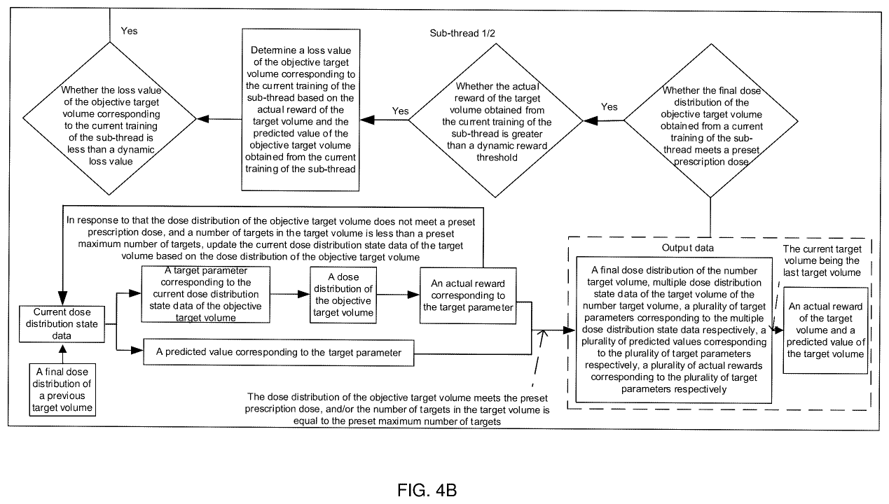

The above-mentioned two methods for training a model in different scenarios provided in some embodiments of the present disclosure are described in combination with FIG. 3A, FIG. 3B, FIG. 4A, and FIG. 4B. FIG. 3A is a flow chart of an exemplary method for training a model implemented in a scenario with a single target volume provided by the present disclosure. FIG. 3B is another flow chart of sub-thread 1 or 2 in FIG. 3A according to some embodiments. As shown in FIG. 3A and FIG. 3B, the method for training a model implemented in a scenario with a single target volume includes the following steps.

In A1, initial dose distribution state data of an objective target volume is acquired.

The specific implementation process of A1 may refer to the description of S201, which will not be repeated herein.

After the initial dose distribution state data of the objective target volume is acquired, the following A2-A7 are executed using each of a plurality of sub-threads based on the initial dose distribution state data of the objective target volume and the current policy data of an actor network and a critic network to acquire output data of each sub-thread of the plurality of sub-threads.

In A2, a target parameter corresponding to the current dose distribution state data of the objective target volume and a predicted value corresponding to the target parameter is determined based on current dose distribution state data of the objective target volume and the current policy data of the actor network and the critic network.

Due to the fact that a generation process of a treatment plan with Gamma knife is a process for placing targets in sequence, a dose distribution state of the objective target volume would change after the placement of a target is completed. Therefore, during the generation process of a complete treatment plan for the objective target volume, the current dose distribution state of the objective target volume would change with the placement of one target after another.

Therefore, after obtaining the current dose distribution state data of the objective target volume (for a first target, the current dose distribution state data of the objective target volume is the initial dose distribution state data of the objective target volume), the actor network included in the deep reinforcement learning model may make an action selection decision based on the current policy data of the actor network and the current dose distribution state data of the objective target volume. That is, the actor network may output an action decision parameter to a sub-thread. The sub-thread, after receiving the action decision parameter sent from the actor network, may determine the target parameter based on the action decision parameter.

In some embodiments, the above target parameter include one of the following target parameters: a size of a target, a position of a target, and a weight of a target. A weight of a target is used to determine a dose of a target.

For example, when the target parameter is a size of a target, the sub-thread may determine a size of a current target based on the action decision parameter sent from the actor network. Then, a target parameter (i.e., a size of a target) corresponding to the current dose distribution state data of the objective target volume can be obtained. Secondly, since the deep reinforcement learning model further includes the critic network, while the actor network selects a target parameter, the critic network may predict a value of the selection action of the target parameter based on the current policy data of the critic network and the current dose distribution state data of the objective target volume. That is, the critic network outputs a predicted decision parameter to a sub-thread. The sub-thread, after receiving the predicted decision parameter sent from the critic network, may determine a predicted value corresponding to the target parameter based on the predicted decision parameter.

In A3, a dose distribution of the objective target volume is determined based on the target parameter corresponding to the current dose distribution state data of the objective target volume.

The dose distribution of the objective target volume refers to a dose distribution of the objective target volume after placing the current target (i.e., a target corresponding to the target parameter determined in step A2) in the objective target volume.

Specifically, after determining the target parameter (e.g., a size of a target) corresponding to the current dose distribution state data of the objective target volume, the sub-thread may call a shape matching algorithm to match the selected size of a target with a contour of the objective target volume, and determine an optimal placement position of the current target within the objective target volume, i.e., a position of the current target. Next, the sub-thread may determine a weight of the current target, based on the size of the current target, the position of the current target, and a preset prescription dose. In some embodiments, after determining a position of a target and a size of the target, the weight of the target may also be defaulted to 1.

It can be understood that when the target parameter is a position of a target or a weight of a target, the sub-thread may call other algorithms (i.e., other algorithms except for the shape matching algorithm) to determine other target parameters (e.g., a size of a target and a weight of a target; a size of a target and a position of a target) included in the treatment plan.

After determining the size of the target, the position of the target, and the weight of the target, the sub-thread may perform a placement action for the target in the objective target volume based on the size of the target, the position of the target, and the weight of the target. After placing the target, the dose distribution of the objective target volume would change. In this case, the sub-thread may determine the dose distribution of the objective target volume.

In A4, an actual reward corresponding to the target parameter is determined based on the dose distribution of the objective target volume.

After determining the dose distribution of the objective target volume, in order to obtain a trained deep reinforcement learning model subsequently, the sub-thread may determine the actual reward corresponding to the target parameter (e.g., the size of the target) under a current dose distribution state based on the dose distribution of the objective target volume.

Due to the fact that the dose distribution of the objective target volume is determined after the current target is placed, the dose distribution of the objective target volume may truly reflect the contribution of the selection action of the target parameter (e.g., the size of the target) in the step A2 to the dose distribution of the objective target volume. Therefore, based on the dose distribution of the objective target volume, the actual reward corresponding to the target parameter (e.g., the size of the target) under the current dose distribution state can be obtained.

For example, the actual reward corresponding to the target parameter includes: a positive reward and a negative reward. Compared the situation before placing the current target with the situation after placing the current target, a value obtained by summing a value obtained by multiplying a growth value of the conformity index of the objective target volume by a corresponding weight with a value obtained by multiplying a growth value of a dose coverage rate of the objective target volume by a corresponding weight may be designated as a positive reward. Compared the situation before placing the current target with the situation after placing the current target, a value obtained by multiplying a growth value of a dose overflow rate of the objective target volume by a corresponding weight may be designated as a negative reward.

In A5, in response to that the dose distribution of the objective target volume does not meet a preset prescription dose, and a number of targets in the objective target volume is less than a preset maximum number of targets, the current dose distribution state data of the objective target volume is updated based on the dose distribution of the objective target volume.

When the dose distribution of the objective target volume does not meet the preset prescription dose, and a number of targets in the objective target volume is less than the preset maximum number of targets, it means that the treatment plan has not been completed in the objective target volume. In this case, the sub-thread may determine the current dose distribution state data of the objective target volume based on the dose distribution of the objective target volume, and update the current dose distribution state data of the objective target volume. That is, the sub-thread updates mask data of the objective target volume, the dose distribution of the objective target volume, a volume of an area in the objective target volume with an insufficient dose distribution, and a volume of an area in the objective target volume with an overflow dose.

Then, the sub-thread repeats steps A2-A4 to determine a subsequent plurality of target parameters, predicted values corresponding to the plurality of target parameters respectively, and actual rewards corresponding to the plurality of target parameters respectively based on the updated current dose distribution state data of the objective target volume, the current policy data of the actor network and the critic network.

In some embodiments, when the sub-thread updates the current dose distribution state data of the objective target volume based on the dose distribution of the objective target volume, the updated current dose distribution state data is obtained by performing a feature extraction on the dose distribution of the objective target volume.

In A6, in response to that the dose distribution of the objective target volume meets the preset prescription dose, and/or the number of targets in the objective target volume is equal to the preset maximum number of targets, the final dose distribution of the objective target volume, the multiple dose distribution state data of the objective target volume, the plurality of target parameters corresponding to the multiple dose distribution state data respectively, the plurality of predicted values corresponding to the plurality of target parameters respectively, and the plurality of actual rewards corresponding to the plurality of target parameters respectively are determined.

Specifically, when the dose distribution of the objective target volume meets the preset prescription dose, and/or a number of targets in the objective target volume is equal to the preset maximum number of targets, it means that the treatment plan has been completed in the objective target volume. In this case, the sub-thread may determine the final dose distribution of the objective target volume, the multiple dose distribution state data of the objective target volume, the plurality of target parameters corresponding to the multiple dose distribution state data respectively, the plurality of predicted values corresponding to the plurality of target parameters respectively, and the plurality of actual rewards corresponding to the plurality of target parameters respectively.

In A7, an actual reward of the objective target volume and a predicted value of the objective target volume are determined based on the final dose distribution of the objective target volume and the plurality of predicted values corresponding to the plurality of target parameters respectively.

Specifically, when the dose distribution of the objective target volume meets the preset prescription dose, and/or a number of targets in the objective target volume is equal to the preset maximum number of targets, the final dose distribution of the objective target volume, and the plurality of predicted values corresponding to the plurality of target parameters respectively may be obtained. In this case, the sub-thread may determine the actual reward of the objective target volume based on the final dose distribution, and determine the predicted value of the objective target volume based on the plurality of predicted values corresponding to the plurality of target parameters respectively.

In some embodiments, after the final dose distribution of the objective target volume is determined, the conformity index of the objective target volume and a dose coverage rate of the objective target volume may be determined based on the final dose distribution of the objective target volume, and the actual reward of the objective target volume may be determined based on the dose coverage rate and the conformity index of the objective target volume.

In some embodiments, after the plurality of predicted values corresponding to the plurality of target parameters respectively are determined, the plurality of predicted values corresponding to the plurality of target parameters respectively may be summed to obtain the predicted value of the objective target volume.

After A2-A7 have been executed by the plurality of sub-threads in parallel, the output data of the plurality of sub-threads has been determined.

After determining the output data of the plurality of sub-threads, since the treatment plan generated by each sub-thread needs to be determined whether the treatment plan is a good treatment plan, that is, the output data obtained from each sub-thread needs to be determined whether the output data of each sub-thread can be used to update the current policy data of the actor network and the critic network. Therefore, the following A8-A10 may be executed sequentially on the output data of each sub-thread, thus completing the current training for the deep reinforcement learning model.

In A8, in response to that the final dose distribution of the objective target volume obtained from a current training for the sub-thread meets a preset prescription dose, whether the actual reward of the objective target volume obtained from the current training for the sub-thread is greater than a dynamic reward threshold is determined.

The dynamic reward threshold is an actual reward of the objective target volume corresponding to output data of the sub-thread used for updating the current policy data of the actor network and the critic network previously. The final dose distribution of the objective target volume is a dose distribution corresponding to the treatment plan of the objective target volume after completing the generation of the treatment plan for the objective target volume.

It should be noted that since the treatment plan for the objective target volume is completed when the dose distribution of the objective target volume meets the preset prescription dose, and/or a number of targets in the objective target volume is equal to the preset maximum number of targets, therefore, if the treatment plan for the objective target volume is obtained in response that the updated dose distribution of the objective target volume meets the preset prescription dose, the sub-thread may not need to determine whether the dose distribution corresponding to the treatment plan meets the preset prescription dose, and can proceed to the subsequent judgment steps. In some embodiments, the sub-thread may also re-determine whether the dose distribution corresponding to the treatment plan meets the preset prescription dose.

However, if the treatment plan for the objective target volume is obtained when a number of targets in the objective target volume is equal to the preset maximum number of targets, since the treatment plan only meets the preset maximum number of targets, but does not necessarily meet the preset prescription dose, the sub-thread needs to determine whether the dose distribution corresponding to the treatment plan meets the preset prescription dose.

If the dose distribution corresponding to the treatment plan does not meet the preset prescription dose, it means that the treatment plan is not a good treatment plan. Therefore, the output data of the sub-thread may be discarded. That is, there is no need to update the current policy data of the actor network and the critic network based on the output data of the sub-thread.

Correspondingly, if the dose distribution corresponding to the treatment plan meets the preset prescription dose, it means that the treatment plan may meet the requirement for meeting the preset prescription dose. In this case, the sub-thread may determine whether the actual reward of the objective target volume is greater than a dynamic reward threshold.

The dynamic reward threshold is an actual reward of the objective target volume corresponding to output data of the sub-thread used for updating the current policy data of the actor network and the critic network previously.

By judging whether the actual reward of the objective target volume obtained from the current training is greater than the dynamic reward threshold, whether the quality of the treatment plan obtained from the current training is better than the quality of the treatment plan used for updating the current policy data of the actor network and the critic network previously may be determined.

When the actual reward of the objective target volume obtained from the current training is less than or equal to the dynamic reward threshold, it means that the quality of the treatment plan obtained from the current training is inferior to or equal to the quality of the treatment plan used for updating the current policy data of the actor network and the critic network previously. Therefore, the output data of the sub-thread may be discarded. That is, there is no need to update the current policy data of the actor network and the critic network based on the output data of the sub-thread.

Correspondingly, when the actual reward of the objective target volume obtained from the current training is greater than the dynamic reward threshold, it means that the quality of the treatment plan obtained from the current training is better than the quality of the treatment plan used for updating the current policy data of the actor network and the critic network previously. That is, the quality of the treatment plan is better. In this case, A9 may be executed by the sub-thread.

In A9, in response to that the actual reward of the objective target volume obtained from the current training for the sub-thread is greater than the dynamic reward threshold, a loss value of the objective target volume corresponding to the current training for the sub-thread based on the actual reward and the predicted value of the objective target volume obtained from the current training for the sub-thread is determined.

Specifically, when the actual reward of the objective target volume obtained from the current training is greater than the dynamic reward threshold, it means that the quality of the treatment plan obtained from the current training is better. In this case, the sub-thread may determine the loss value of the objective target volume corresponding to the current training based on the actual reward of the objective target volume and the predicted value of the objective target volume obtained from the current training.

In some embodiments, the sub-thread may use a preset loss function to determine the loss value of the objective target volume corresponding to the current training based on the actual reward of the objective target volume and the predicted value of the objective target volume obtained from the current training.

In some embodiments, the preset loss function may be a relative advantage function or another general loss function, which is not limited in the present disclosure.

Then, the sub-thread may determine whether the loss value of the objective target volume corresponding to the current training is less than a dynamic loss value.

The dynamic loss value is a loss value of the objective target volume corresponding to output data of the sub-thread used for updating the current policy data of the actor network and the critic network previously.

If the loss value of the objective target volume corresponding to the current training is greater than or equal to the loss value of the objective target volume corresponding to output data of the sub-thread used for updating the current policy data of the actor network and the critic network previously, it means that the deep reinforcement learning model is inaccurate for predicting the quality of the treatment plan. Therefore, the output data of the sub-thread may be discarded. That is, there is no need to update the current policy data of the actor network and the critic network based on the output data of the sub-thread.

If the loss value of the objective target volume corresponding to the current training is less than the loss value of the objective target volume corresponding to output data of the sub-thread used for updating the current policy data of the actor network and the critic network previously, it means that the deep reinforcement learning model is accurate for predicting the quality of the treatment plan. In this case, A10 may be executed by the sub-thread.

In A10, in response to that the loss value of the objective target volume corresponding to the current training for the sub-thread is less than a dynamic loss value, the current policy data of the actor network and the critic network is updated based on multiple dose distribution state data obtained from the current training for the sub-thread, a plurality of target parameters corresponding to the multiple dose distribution state data respectively, a plurality of predicted values corresponding to the plurality of target parameters respectively, a plurality of actual rewards corresponding to the plurality of target parameters respectively, and the actual reward of the objective target volume.

In some embodiments, the method of updating the current policy data of the actor network and the critic network based on the multiple dose distribution state data obtained from the current training for the sub-thread, the plurality of target parameters corresponding to the multiple dose distribution state data respectively, the plurality of predicted values corresponding to the plurality of target parameters respectively, the plurality of actual rewards corresponding to the plurality of target parameters respectively, and the actual reward of the objective target volume specifically includes:
determining an actual cumulated reward value of the plurality of target parameters based on the plurality of actual rewards corresponding to the plurality of target parameters respectively and the actual reward of the objective target volume, and updating the current policy data of the actor network based on the multiple dose distribution state data, the plurality of target parameters corresponding to the multiple dose distribution state data respectively, and the actual cumulated reward value of the plurality of target parameters, and updating the current policy data of the critic network based on the multiple dose distribution state data, the plurality of target parameters corresponding to the multiple dose distribution state data respectively, and the plurality of predicted values corresponding to the plurality of target parameters respectively.

Specifically, the quality of the treatment plan for the objective target volume is a result of the cooperation of the plurality of target parameters selected by the actor network corresponding to the multiple dose distribution state data. In a certain dose distribution state of the objective target volume, an actual reward of a target parameter selected by the actor network being high does not mean that the quality of the treatment plan for the objective target volume is good. If the quality of a final treatment plan for the objective target volume is good, a reference value of the actor network for the selection of the target parameter may be great. If the quality of a final treatment plan for the objective target volume is not good, a reference value of the actor network for the selection of the target parameter may be decreased. Therefore, after obtaining the plurality of target parameters corresponding to the multiple dose distribution state data respectively, the actual cumulated reward value of the plurality of target parameters may also be determined based on the plurality of actual rewards corresponding to the plurality of target parameters respectively and the actual reward of the objective target volume.

The actual cumulated reward value may include an actual reward of each target parameter selected by the actor network, and a delayed reward value obtained after the completion of the entire treatment plan. The delayed reward value is determined based on the actual reward of the objective target volume. That is, after the treatment plan for the objective target volume is completed, the actual reward of the objective target volume is determined based on the dose distribution of the treatment plan, and the actual reward may be assigned to target parameters selected by the actor network in each target parameter combination that constitutes the treatment plan according to corresponding weights, thereby forming delayed reward values of the target parameters selected by the actor network. The actual reward and the delayed reward value of each target parameter selected by the actor network may be accumulated to obtain the actual cumulated reward value of each target parameter selected by the actor network.

When the actual cumulated reward value is relatively high, it means that a reference value of the actor network for the selection of the target parameter is great. In this case, the current policy data of the actor network is updated based on the multiple dose distribution state data, the plurality of target parameters corresponding to the multiple dose distribution state data respectively, and the actual cumulated reward value of the plurality of target parameters.

Meanwhile, since the loss value of the objective target volume is obtained based on the predicted value of the objective target volume, and the predicted value of the objective target volume is an accumulation of the predicted values of the plurality of target parameters selected by the actor network, when the loss value of the objective target volume is less than the dynamic loss value, it means that the predicted values of the plurality of target parameters selected by the actor network have a high reference value. In this case, the current policy data of the critic network can be updated based on the multiple dose distribution state data, the plurality of target parameters corresponding to the multiple dose distribution state data respectively, and the plurality of predicted values corresponding to the plurality of target parameters respectively.

In some embodiments, the method of updating the current policy data of the actor network and the critic network based on the multiple dose distribution state data obtained from the current training for the sub-thread, the plurality of target parameters corresponding to the multiple dose distribution state data respectively, the plurality of predicted values corresponding to the plurality of target parameters respectively, the plurality of actual rewards corresponding to the plurality of target parameters respectively, and the actual reward of the objective target volume specifically includes:
updating the current policy data of the actor network and the critic network by using a proximal policy optimization algorithm based on the multiple dose distribution state data obtained from the current training for the sub-thread, the plurality of target parameters corresponding to the multiple dose distribution state data respectively, the plurality of predicted values corresponding to the plurality of target parameters respectively, the plurality of actual rewards corresponding to the plurality of target parameters respectively, and the actual reward of the objective target volume.

The proximal policy optimization (PPO) algorithm may be used for training a model efficiently in a large-scale environment, and may be used to handle continuous action space and high-dimensional state space, which is in line with the characteristics of the design for the treatment plan with Gamma knife. Thus, the trial and error process may be performed repeatedly, thus reducing the dependence on clinical experience, and further, improving the efficiency of designing a treatment plan by a physician.

It can be understood that other algorithms (e.g., the advantage actor critic (A2C) algorithm, and the trust region policy optimization (TPRO) algorithm) may also be used to update the current policy data of the actor network and the critic network.

In A11, the training processes A1-A10 are iterated until a count of iterating the training process reaches a preset count, so as to obtain the deep reinforcement learning model that has been trained.

The detailed description of the operation A11 may refer to the description of operation S204, which will not be repeated herein.

FIG. 3A illustrates a method for training a model implemented in a scenario with a single target volume, and FIG. 3B is another flow chart of sub-thread 1 or 2 in FIG. 3A according to some embodiments. However, a situation that a to-be-treated subject includes multiple target volumes is common in the clinical radiotherapy. Therefore, FIG. 4A illustrates a method for training a model implemented in a scenario with multiple target volumes provided by the present disclosure. FIG. 4B is another flow chart of sub-thread 1 or 2 in FIG. 4A according to some embodiments. As shown in FIG. 4A and FIG. 4B, the method for training a model implemented in a scenario with multiple target volumes specifically includes the following steps.

In B1, initial dose distribution state data of an objective target volume is acquired.

The specific implementation process of this step may refer to the description of S201, which will not be repeated herein.

After the initial dose distribution state data of the objective target volume is acquired, the following B2-B8 are executed using each sub-thread of a plurality of sub-threads based on the initial dose distribution state data of the objective target volume and the current policy data of an actor network and a critic network to acquire output data of each of the plurality of sub-threads.

In B2, a target parameter corresponding to the current dose distribution state data of the current target volume and a predicted value corresponding to the target parameter are determined based on current dose distribution state data of a current target volume and the current policy data of the actor network and the critic network.

It should be noted that if a current target volume is the first target volume, the current dose distribution state data of the current target volume is the initial dose distribution state data acquired in B1.

If the current target volume is not the first target volume, due to the interaction between adjacent target volumes, each sub-thread may designate the dose distribution state data corresponding to the final dose distribution of a previous target volume determined as the current dose distribution state data of the current target volume.

The specific implementation process of this step may refer to the description of A2, which will not be repeated herein.

In B3, a dose distribution of the current target volume is determined based on the target parameter corresponding to the current dose distribution state data of the current target volume.

The specific implementation process of this step may refer to the description of A3, which will not be repeated herein.

In B4, an actual reward corresponding to the target parameter is determined based on the dose distribution of the current target volume.

The specific implementation process of this step may refer to the description of A4, which will not be repeated herein.

In B5, in response to that the dose distribution of the current target volume does not meet a preset prescription dose, and a number of targets in the current target volume is less than a preset maximum number of targets, the current dose distribution state data of the current target volume is updated based on the dose distribution of the current target volume.

The specific implementation process of this step may refer to the description of A5, which will not be repeated herein.

In B6, in response to that the dose distribution of the current target volume meets the preset prescription dose, and/or the number of targets in the current target volume is equal to the preset maximum number of targets, a final dose distribution of the current target volume, a plurality of dose distribution state data of the current target volume, a plurality of present parameters corresponding to the plurality of dose distribution state data respectively, a plurality of predicted values corresponding to the plurality of target parameters respectively, and a plurality of actual rewards, corresponding to the plurality of target parameters respectively are determined, and an actual reward of the current target volume and a predicted value of the current target volume are determined based on the final dose distribution of the current target volume and the plurality of predicted values corresponding to the plurality of target parameters respectively.

The specific implementation process of this step may refer to the description of A6, which will not be repeated herein.

After the actual reward and the predicted value of the current target volume are determined, as the objective target volume includes multiple target volumes, a sub-thread may need to determine whether the current target volume is the last target volume.

In B7, in response to that the current target volume is not the last target volume, the current target volume is updated, and the current dose distribution state data of the current target volume is determined based on a final dose distribution of a previous target volume of the current target volume.

In B8, in response to that the current target volume is the last target volume, the actual reward and the predicted value of the objective target volume are determined based on the final dose distribution of each target volume of the multiple target volumes and the predicted value of each target volume of the multiple target volumes.

Specifically, after determining the actual reward and the predicted value of a target volume, the sub-thread may perform the same operations (i.e., iterating steps B2-B6) on a next target volume until an actual reward and a predicted value of the last target volume of the multiple target volumes is determined.

The actual reward of the multiple target volumes may be an overall reward for the selection of the plurality of target parameters of the multiple target volumes obtained after completing the above iteration. For example, a number of the multiple target volumes is three, after determining the plurality of target parameters of the third target volume, all target parameters of the three target volumes are determined as a group of selection, and this group of selection may be rewarded as a whole.

Therefore, the actual reward of the objective target volume may be jointly determined by the sub-threads based on the final dose distribution of the multiple target volumes. That is, different weights may be assigned to the actual rewards of different target volumes based on the importance of the different target volumes, and further, the actual rewards of different target volumes may be weighted and summed to obtain the actual reward of the objective target volume.

The predicted value of the objective target volume may be determined by summing the predicted values of the multiple target volumes by the sub-threads.

Hereto, the output data of the plurality of sub-threads is determined.

After the output data of the plurality of sub-threads is determined, since the treatment plan generated by each sub-thread needs to be determined whether the treatment plan is a good treatment plan, that is, the output data obtained from each sub-thread needs to be determined whether the output data of each sub-thread can be used to update the current policy data of the actor network and the critic network. Therefore, the following B9-B11 may be executed sequentially on the output data of each sub-thread, thus completing the current training for the deep reinforcement learning model.

In B9, in response to that the final dose distribution of the objective target volume obtained from a current training for the sub-thread meets a preset prescription dose, whether the actual reward of the objective target volume obtained from the current training for the sub-threads is greater than a dynamic reward threshold is determined.

The specific implementation process of this step may refer to the description of A8, which will not be repeated herein.

It should be noted that due to a number of the objective target volumes being multiple, when the final dose distribution of each target volume of the multiple target volumes meets the preset prescription dose, the sub-thread may determine that the final dose distribution of the objective target volume obtained from the current training meets the preset prescription dose.

In B10, in response to that the actual reward of the objective target volume obtained from the current training for the sub-thread is greater than the dynamic reward threshold, a loss value of the objective target volume corresponding to the current training for the sub-thread is determined based on the actual reward and the predicted value of the objective target volume obtained from the current training for the sub-thread.

The specific implementation process of this step may refer to the description of A9, which will not be repeated herein.

In B11, in response to that the loss value of the objective target volume corresponding to the current training for the sub-threads is less than a dynamic loss value, the current policy data of the actor network and the critic network is updated based on multiple dose distribution state data obtained from the current training for the sub-thread, a plurality of target parameters corresponding to the multiple dose distribution state data respectively, a plurality of predicted values corresponding to the plurality of target parameters respectively, a plurality of actual rewards corresponding to the plurality of target parameters respectively, and the actual reward of the objective target volume.

The specific implementation process of this step may refer to the description of A10, which will not be repeated herein.

In B12, a trained deep reinforcement learning model is obtained by iterating the training processes B1-B11 until a count of iterating the training process reaches a preset count.

The specific implementation process of this step may refer to the description of S204, which will not be repeated herein.

Next, functions of a processor for generating a treatment plan are introduced as follows.

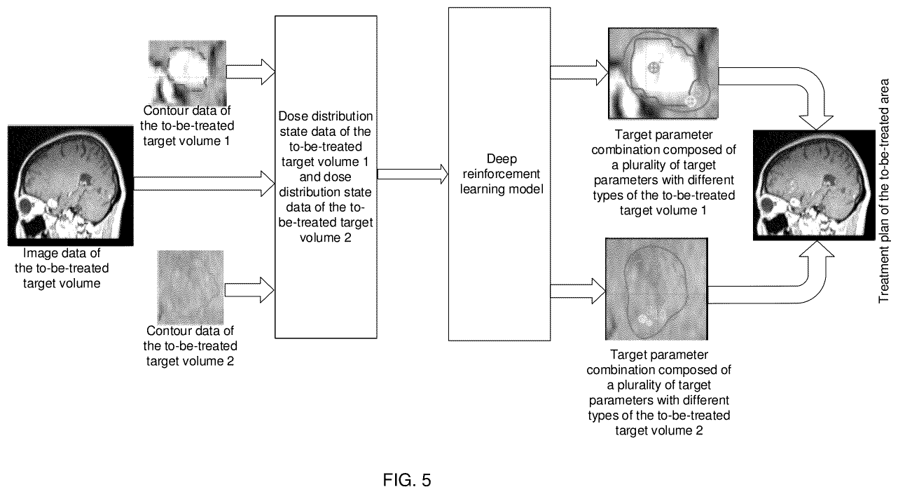

FIG. 5 is a flow chart of an exemplary method for generating a treatment plan by a processor according to some embodiments. As shown in FIG. 5, the method includes S501, S502, S503, and S504.

In S501, image data of a to-be-treated target volume and contour data of the to-be-treated target volume are acquired.

A specific process of acquiring the image data and the contour data of the to-be-treated objective target volume may refer to a specific process of acquiring the image data and the contour data of the objective target volume in S201, which will not be repeated herein.

For example, as shown in FIG. 5, the image data of the to-be-treated target volume may be a CT image of the head of the to-be-treated target volume, and the contour data may be contour data of target volume 1 and target volume 2 in the head of the to-be-treated target volume.

In S502, dose distribution state data of the to-be-treated target volume is determined based on the image data and the contour data of the to-be-treated target volume.

A specific process of determining the dose distribution state data of the to-be-treated target volume may refer to specific process of determining the initial dose distribution state data of the to-be-treated target volume in S201, which will not be repeated herein.

In S503, the dose distribution state data of the to-be-treated target volume is inputted into a deep reinforcement learning model, so as to obtain a target parameter of the to-be-treated target volume.

The deep reinforcement learning model is obtained based on the method for training the model described in FIG. 2, FIG. 3A and FIG. 3B, or FIG. 4A and FIG. 4B.

For example, as shown in FIG. 5, the target parameter of the to-be-treated target volume output by the deep reinforcement learning model may include target parameters of the to-be-treated target volume 1 and target parameters of the to-be-treated target volume 2. For example, a target parameter of the to-be-treated target volume output by the deep reinforcement learning model is a size of a target.

In S504, a treatment plan for the to-be-treated target volume is generated based on the target parameter.

For example, as shown in FIG. 5, after determining the target parameters of the to-be-treated target volume 1 and the target parameters of the to-be-treated target volume 2, a processor may determine a target parameter combination 1 composed of a plurality of different types of target parameters of to-be-treated target volume 1 and a target parameter combination 2 composed of a plurality of different types of the target parameters of the to-be-treated target volume 2 based on the target parameters of the to-be-treated target volume 1 and the target parameters of the to-be-treated target volume 2. Then, the processor may generate a treatment plan for the to-be-treated target volume based on the target parameter combination 1 and the target parameter combination 2.

A target parameter combination includes: a size of a target, a position of a target, and a weight of a target.

Taking the target parameter being a size of a target as an example, a process of generating a treatment plan by a processor is described as follows. It can be understood that the target parameter may also be a position of a target or a weight of a target.

Firstly, the processor acquires image data and contour data of the to-be-treated target volume. Then, the processor inputs the acquired image data and contour data into the deep reinforcement learning model trained by using the method for training the deep reinforcement learning model shown in FIG. 2, FIG. 3A and FIG. 3B, or FIG. 4A and FIG. 4B, to obtain a size of a first target output by the deep reinforcement learning model. Then, a thread in the processor calls a shape matching algorithm to match the size of the first target output by the deep reinforcement learning model with a contour of the to-be-treated target volume, and determines an optimal position for placing the first target with the size of the first target within the to-be-treated target volume. That is, a position of the first target is determined. Then, the thread in the processor calls a weight optimization algorithm to determine a weight of the first target based on the size of the first target and the position of the target that have been determined, and a prescription dose. Hereto, a target parameter combination for the first target of the treatment plan is determined.

Then, the thread in the processor calculates the dose distribution of the to-be-treated target volume based on the target parameter combination of the first target, and determines the current dose distribution state data of the to-be-treated target volume based on the dose distribution of the to-be-treated target volume. Then, the current dose distribution state data of the to-be-treated target volume is input into the deep reinforcement learning model to obtain a size of a second target output by the deep reinforcement learning model, a target parameter combination for the second target is determined based on the size of the second target (the method for determining a position and a weight of the second target is the same as the method for determining a position and a weight of the first target, which will not be repeated herein).

Iterating the processes of determining a size, a position, and a weight of a target mentioned above to determine target parameter combinations for remaining targets until the dose distribution of the to-be-treated target volume meets the prescription dose, and/or a number of targets in the to-be-treated target volume is equal to a preset maximum number of targets, then the target parameter combination for the to-be-treated target volume is determined. The target parameter combination for the to-be-treated target volume includes a target parameter combination of a plurality of targets. That is, the target parameter combination includes a plurality of sets composed of a size(S) of a target, a position (P) of a target, and a weight (W) of a target, such as {(S1, P1, W1), (S2, P2, W2), . . . (Sn, Pn, Wn)}.

It can be understood that a target weight of a target mentioned above may also be obtained without calling an algorithm, and may be defaulted to 1.

The treatment plan may be displayed in a displayed form of an image corresponding to image data of a to-be-treated subject. In the image corresponding to the image data of the to-be-treated subject, the target parameter combination of the target volume 1 and the target parameter combination of the target volume 2 may be clearly labeled.

In some embodiments, after determining the treatment plan for the to-be-treated target volume, a physician may also manually adjust the target parameter combination for the treatment plan for the to-be-treated target volume based on experience, and thus, the treatment plan for the to-be-treated target volume is determined based on the adjusted target parameter combination.

The above descriptions mainly involves the methods provided in some embodiments of the present disclosure from the perspective of a computer system. In order to achieve the above functions, the computer system includes hardware structures and/or software modules configure to execute the functions. Those skilled persons in the art should easily realize that the embodiments of present disclosure can be implemented in the form of a hardware or a combination of hardware and computer software in combination with the units and algorithm steps described in the embodiments of the present disclosure. Whether a certain function is executed by hardware or computer software depends on the specific application and design constraints of the technical solution. Professional technicians may use different methods to implement the described functions for each specific application, but such implementation should not be considered beyond the scope of the present disclosure.

The embodiments of the present disclosure may be divided into functional modules based on the computer system. For example, functional modules may be divided corresponding to different functions, or two or more functions may be integrated into one processing module. An integrated module mentioned above may be implemented in both hardware and software functional modules. In some embodiments, the modules in the embodiments of the present disclosure may be divided for illustration and may merely serve as a logical functional division. In the actual implementation, the modules in the present disclosure may be divided by other methods.

In some embodiments of the present disclosure, an electronic device is further provided. The electronic device includes at least one processor, and a memory connected in communication with the at least one processor. The memory stores instructions that can be executed by the at least one processor, and the instructions are executed by the at least one processor, such that the at least one processor can execute the method for generating the treatment plan provided by the present disclosure.

In some embodiments of the present disclosure, a non-transitory computer readable storage medium stored computer instructions thereon is further provided. The computer instructions are used to cause the electronic device to implement the method for training a deep reinforcement learning model for generating a treatment plan or the method for generating a treatment plan.

In some embodiments of the present disclosure, a computer program product is further provided. The computer program product includes a computer program. The computer program, upon being executed by a processor, implements the method for training a deep reinforcement learning model for generating a treatment plan or the method for generating a treatment plan.

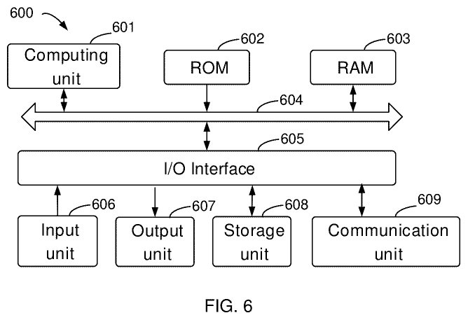

In some embodiments, the electronic device may be the electronic device a2 or the electronic device b2 shown in FIG. 1. FIG. 6 is a block diagram of an example electronic device 600 that can be configured to implement some embodiment of the present disclosure. The electronic device 600 is intended to represent various forms of digital computers, such as a laptop, a desktop computer, a workstation, a personal digital assistant, a server, a blade server, a mainframe computer, or other suitable computers. The electronic device 600 may also represent various forms of mobile devices, such as a personal digital assistant, a cellular phone, a smartphone, a wearable device, or other similar computing devices. The components, connections and relationships between the components herein, and functions of the components are merely illustrative, and are not intended to limit the scope of the present disclosure.

As shown in FIG. 6, the electronic device 600 may include a computing unit 601, which may perform various appropriate actions and processing based on computer programs stored in a read only memory (ROM) 602 or computer programs loaded from a storage unit 608 into a random access memory (RAM) 603. In RAM 603, various programs and data required for the operation of the electronic device 600 may also be stored. The computing unit 601, the ROM 602, and the RAM 603 are connected to each other through a bus 604. The input/output (I/O) interface 605 may also connected to the bus 604.

A plurality of components in the electronic device 600 may be connected to the I/O interface 605, including: an input unit 606 (e.g., a keyboard, a mouse, etc.), an output unit 607 (e.g., various types of displays, speakers, etc.), a storage unit 608 (e.g., a magnetic disk, an optical disc, etc.), and a communication unit 609 (e.g., a network card, a modem, a wireless communication transceiver, etc.). The communication unit 609 allows the electronic device 600 to exchange information/data with other devices through computer networks, such as the Internet and/or various telecommunications networks.

The computing unit 601 may be various general and/or specialized processing components with processing and computing capabilities. The computing unit 601 includes, but are not be limited to, a central processing unit (CPU), a graphics processing units (GPU), various specialized artificial intelligence (AI) computing chips, various computing units for running machine learning model algorithms, a digital signal processor (DSP), and any appropriate processor, a controller, or a microcontroller, etc. The computing unit 601 executes various methods and processes described above, such as the method for generating a treatment plan. For example, in some embodiments, the method for generating a treatment plan may be implemented as a computer software program, which is tangibly included in a machine readable medium, such as the storage unit 608. In some embodiments, some or all of the computer programs may be loaded and/or installed on the electronic device 600 via the ROM 602 and/or the communication unit 609. When a computer program is loaded into the RAM 603 and executed by the computing unit 601, one or more steps of the method for generating a treatment plan described above may be executed. In other embodiments, the computing unit 601 may be configured to execute the method for generating a treatment plan by any other appropriate means (e.g., by virtue of firmware).

The various implementations of systems and technologies described above in the present disclosure could be implemented in a digital electronic circuit system, an integrated circuit system, a field programmable gate array (FPGA), an application specific integrated circuit (ASIC), an application specific standard part (ASSP), a system on chip (SOC) systems, a complex programmable logic device (CPLD), a computer hardware, firmware, software, and/or any combination thereof. These various implementation methods may include: implementation in one or more computer programs, which could be executed and/or interpreted on a programmable system including at least one programmable processor. The programmable processor may be a dedicated or general-purpose programmable processor that can receive data and instructions from a storage system, at least one input device, and at least one output device, and transmit data and instructions to the storage system, the at least one input device, and the at least one output device.

The program code for implementing the method described in the present disclosure may be written in any combination of one or more programming languages. These program codes may be provided to a processor or a controller of a general-purpose computer, a specialized computer, or other programmable data processing devices, so that the program code, when executed by the processor or the controller, enables the functions/operations specified in the flow chart and/or block diagram to be implemented. The program codes may be completely executed on the machine, partially executed on the machine, as an independent software package partially executed on the machine, and partially executed on remote machines or completely executed on remote machines or servers.

In the context of the present disclosure, a machine readable medium may be a tangible medium that can contain or store programs for use by or in combination with instruction execution systems, apparatuses, or devices. The machine readable medium may be machine readable signal medium or machine readable storage medium. The machine readable medium may include, but is not limited to an electronic, a magnetic, an optical, an electromagnetic, an infrared, or a semiconductor system, apparatus, or device, or any combination thereof. More specific examples of machine readable storage medium include an electrical connection based on one or more wires, a portable computer disk, a hard drive, a random access memory, a read-only memory (ROM), an erasable programmable read only memory (EPROM or flash memory), an optical fiber, a compact disc read only memory (CD-ROM), an optical storage device, a magnetic storage device, or any combination thereof.

In order to provide interaction with users, the system and technology described in the present disclosure can be implemented on a computer, which includes a display device (e.g., a cathode ray tube (CRT) or a liquid crystal display (LCD)) for displaying information to users. The computer may further include a keyboard and a pointing device (e.g., a mouse or trackball). Thus, the users can provide input to the computer via the keyboard and the pointing device. Other types of devices may also be used to provide interaction with users. For example, the feedback provided to users may be any form of sensory feedback (e.g., visual feedback, auditory feedback, or tactile feedback). Further, the input from the users can be received in any form (including voice input, voice input, or tactile input).

The system and technology described in the present disclosure may be implemented in a computing system that includes a background component (e.g., serving as a data server), or in a computing system that includes a middleware component (e.g., an application server), or in a computing system that includes a front-end component (e.g., a user computer with a graphical user interface or web browser through which users can interact with the implementation of the system and technology described in the present disclosure), or in a computing system that includes any combination of the background component, the middleware components, or the front-end component. The components of the system may be interconnected through any form or medium of digital data communication (e.g., a communication network). Examples of the communication network include: a local area network (LAN), a wide area network (WAN), or the Internet.

A computer system may include both clients and servers. The client and server are generally far away from each other and typically interact through the communication network. A client-server relationship is generated by running computer programs on corresponding computers that have client-server relationships with each other. The server may be a cloud server, a distributed system server, or a server that combines block chain.

It should be understood that various forms of processes shown in the present disclosure may be reordered, added, or deleted. For example, the steps described in the present disclosure may be executed in parallel, in sequence, or in different orders as long as the expected result of the technical solution related to the present disclosure can be achieved, which may not be limited in the present disclosure.

The specific implementations do not limit the scope of the present disclosure. Those skilled in the art should understand that various modifications, combinations, sub-combinations, and substitutions may be made based on design requirements and other factors. Any modifications, equivalent substitutions, and improvements made within the principles of the present disclosure shall be included within the scope of the present disclosure.

What is claimed is:

1. A method for training a deep reinforcement learning model for generating a treatment plan, wherein the deep reinforcement learning model is configured to comprise an actor network and a critic network, and the method comprises:
performing a training process, the training process including the following operations:
acquiring initial dose distribution state data of an objective target volume;
determining, based on the initial dose distribution state data of the objective target volume and current policy data of the actor network and the critic network, output data of each sub-thread of a plurality of sub-threads in parallel by using the plurality of sub-threads; and
updating the current policy data of the actor network and the critic network based on the output data of each sub-thread of the plurality sub-threads in sequence, so as to complete a current training for the deep reinforcement learning model; and
iterating the training process until a count of training the deep reinforcement learning model reaches a preset count, so as to obtain the deep reinforcement learning model that has been trained;
wherein the output data of the sub-thread comprises: a final dose distribution of the objective target volume, multiple dose distribution state data of the objective target volume, a plurality of target parameters corresponding to the multiple dose distribution state data respectively, a plurality of predicted values corresponding to the plurality of target parameters respectively, a plurality of actual rewards corresponding to the plurality of target parameters respectively, a predicted value of the objective target volume, and an actual reward of the objective target volume.

2. The method according to claim 1, wherein when a number of the objective target volume is one, the determining, based on the initial dose distribution state data of the objective target volume and the current policy data of the actor network and the critic network, the output data of the plurality of sub-threads in parallel by using the plurality of sub-threads comprises:
for each sub-thread of the plurality sub-threads, performing the following operations:
determining, based on current dose distribution state data of the objective target volume and the current policy data of the actor network and the critic network, a target parameter corresponding to the current dose distribution state data of the objective target volume and a predicted value corresponding to the target parameter;
determining, based on the target parameter corresponding to the current dose distribution state data of the objective target volume, a dose distribution of the objective target volume;
determining, based on the dose distribution of the objective target volume, an actual reward corresponding to the target parameter;
in response to that the dose distribution of the objective target volume does not meet a preset prescription dose, and a number of targets in the objective target volume is less than a preset maximum number of targets, updating the current dose distribution state data of the objective target volume based on the dose distribution of the objective target volume; or, in response to that the dose distribution of the objective target volume meets the preset prescription dose, and/or the number of targets in the objective target volume is equal to the preset maximum number of targets, determining the final dose distribution of the objective target volume, the multiple dose distribution state data of the objective target volume, the plurality of target parameters corresponding to the multiple dose distribution state data respectively, the plurality of predicted values corresponding to the plurality of target parameters respectively, and the plurality of actual rewards corresponding to the plurality of target parameters respectively; and
determining, based on the final dose distribution of the objective target volume and the plurality of predicted values corresponding to the plurality of target parameters respectively, the predicted value of the objective target volume and the actual reward of the objective target volume.

3. The method according to claim 1, wherein when a number of the objective target volume is multiple, the determining, based on the initial dose distribution state data of the objective target volume and the current policy data of the actor network and the critic network, the output data of the plurality of sub-threads in parallel by using the plurality of sub-threads comprises:
for each sub-thread of the plurality sub-threads, performing the following operations:
determining, based on current dose distribution state data of a current target volume and the current policy data of the actor network and the critic network, a target parameter corresponding to the current dose distribution state data of the current target volume and a predicted value corresponding to the target parameter;
determining, based on the target parameter corresponding to the current dose distribution state data of the current target volume, a dose distribution of the current target volume;
determining, based on the dose distribution of the current target volume, an actual reward corresponding to the target parameter;
in response to that the dose distribution of the current target volume does not meet a preset prescription dose, and a number of targets in the current target volume is less than a preset maximum number of targets, updating the current dose distribution state data of the current target volume based on the dose distribution of the current target volume; or, in response to that the dose distribution of the current target volume meets the preset prescription dose, and/or the number of targets in the current target volume is equal to the preset maximum number of targets, determining a final dose distribution of the current target volume, multiple dose distribution state data of the current target volume, a plurality of target parameters corresponding to the multiple dose distribution state data respectively, a plurality of predicted values corresponding to the plurality of target parameters respectively, and a plurality of actual rewards corresponding to the plurality of target parameters respectively;
determining, based on the final dose distribution of the current target volume and the plurality of predicted values corresponding to the plurality of target parameters respectively, an actual reward of the current target volume and a predicted value of the current target volume;
in response to that the current target volume is not a last target volume, updating the current target volume, and determining the current dose distribution state data of the current target volume based on a final dose distribution of a previous target volume of the current target volume; or, in response to that the current target volume is the last objective target volume, determining the predicted value of the objective target volume and the actual reward of the objective target volume based on a final dose distribution of each target volume of the multiple target volumes and a predicted value of each target volume of the multiple target volumes.

4. The method according to claim 1, wherein the updating the current policy data of the actor network and the critic network based on the output data of each sub-thread of the plurality sub-threads in sequence comprises:
performing the following operations on the output data of each sub-thread of the plurality sub-threads in sequence:
in response to that the final dose distribution of the objective target volume obtained from a current training for the sub-thread meets a preset prescription dose, determining whether the actual reward of the objective target volume obtained from the current training for the sub-thread is greater than a dynamic reward threshold, wherein the dynamic reward threshold is an actual reward of the objective target volume corresponding to output data of the sub-thread used for updating the current policy data of the actor network and the critic network previously;
in response to that the actual reward of the objective target volume obtained from the current training for the sub-thread is greater than the dynamic reward threshold, determining a loss value of the objective target volume corresponding to the current training for the sub-thread based on the actual reward and the predicted value of the objective target volume obtained from the current training for the sub-thread;
in response to that the loss value of the objective target volume corresponding to the current training for the sub-thread is less than a dynamic loss value, updating the current policy data of the actor network and the critic network based on multiple dose distribution state data obtained from the current training for the sub-thread, a plurality of target parameters corresponding to the multiple dose distribution state data respectively, a plurality of predicted values corresponding to the plurality of target parameters respectively, a plurality of actual rewards corresponding to the plurality of target parameters respectively, and the actual reward of the objective target volume, wherein the dynamic loss value is a loss value of the objective target volume corresponding to output data of the sub-thread used for updating the current policy data of the actor network and the critic network previously.

5. The method according to claim 4, wherein the updating the current policy data of the actor network and the critic network based on the multiple dose distribution state data obtained from the current training for the sub-thread, the plurality of target parameters corresponding to the multiple dose distribution state data respectively, the plurality of predicted values corresponding to the plurality of target parameters respectively, the plurality of actual rewards corresponding to the plurality of target parameters respectively, and the actual reward of the objective target volume comprises:
updating the current policy data of the actor network and the critic network by using a proximal policy optimization algorithm based on the multiple dose distribution state data obtained from the current training for the sub-thread, the plurality of target parameters corresponding to the multiple dose distribution state data respectively, the plurality of predicted values corresponding to the plurality of target parameters respectively, the plurality of actual rewards corresponding to the plurality of target parameters respectively, and the actual reward of the objective target volume.

6. The method according to claim 1, wherein the target parameter include one of the following target parameters: a size of a target, a position of a target, and a weight of a target.

7. The method according to claim 1, wherein the dose distribution state data comprises: mask data of the objective target volume, a dose distribution of the objective target volume, a volume of an area in the objective target volume with an insufficient dose distribution, and a volume of an area in the objective target volume with an overflow dose.

8. A method for generating a treatment plan, comprising:
acquiring image data of a to-be-treated target volume and contour data of the to-be-treated target volume;
determining, based on the image data and the contour data, dose distribution state data of the to-be-treated target volume;
inputting the dose distribution state data of the to-be-treated target volume into a deep reinforcement learning model, so as to obtain a target parameter of the to-be-treated target volume, wherein the deep reinforcement learning model is configured to comprise an actor network and a critic network, and the deep reinforcement learning model is trained by the following operations:
performing a training process, the training process including the following operations:
acquiring initial dose distribution state data of an objective target volume;
determining, based on the initial dose distribution state data of the objective target volume and current policy data of the actor network and the critic network, output data of each sub-thread of a plurality of sub-threads in parallel by using the plurality of sub-threads; and
updating the current policy data of the actor network and the critic network based on the output data of each sub-thread of the plurality sub-threads in sequence, so as to complete a current training for the deep reinforcement learning model; and
iterating the training process until a count of training the deep reinforcement learning model reaches a preset count, so as to obtain the deep reinforcement learning model that has been trained;
wherein the output data of the sub-thread comprises: a final dose distribution of the objective target volume, multiple dose distribution state data of the objective target volume, a plurality of target parameters corresponding to the multiple dose distribution state data respectively, a plurality of predicted values corresponding to the plurality of target parameters respectively, a plurality of actual rewards corresponding to the plurality of target parameters respectively, a predicted value of the objective target volume, and an actual reward of the objective target volume;
generating the treatment plan for the to-be-treated target volume based on the target parameter.

9. A non-transitory computer readable storage medium having stored a computer program thereon, wherein the computer program is used to implement a deep reinforcement learning model training method, wherein the deep reinforcement learning model includes an actor network and a critic network, and the method comprises:
performing a training process, the training process including the following operations:
acquiring initial dose distribution state data of an objective target volume;
determining, based on the initial dose distribution state data of the objective target volume and current policy data of the actor network and the critic network, output data of each sub-thread of a plurality of sub-threads in parallel by using the plurality of sub-threads; and
updating the current policy data of the actor network and the critic network based on the output data of each sub-thread of the plurality sub-threads in sequence, so as to complete a current training for the deep reinforcement learning model; and
iterating the training process until a count of training the deep reinforcement learning model reaches a preset count, so as to obtain the deep reinforcement learning model that has been trained;
wherein the output data of the sub-thread comprises: a final dose distribution of the objective target volume, multiple dose distribution state data of the objective target volume, a plurality of target parameters corresponding to the multiple dose distribution state data respectively, a plurality of predicted values corresponding to the plurality of target parameters respectively, a plurality of actual rewards corresponding to the plurality of target parameters respectively, a predicted value of the objective target volume, and an actual reward of the objective target volume.
